(19) United States
(12) Patent Application Publication
(10) Pub. No.: US 2023/0118007 A1
(43) Pub. Date: Apr. 20, 2023

(54) MEDICAL DEVICES AND SYSTEMS FOR USE

(71) Applicant: Synovent Laboratories, LLC, Wilmington, NC (US)

(72) Inventor: Dominic Ventura, Wilmington, NC (US)

(21) Appl. No.: 17/503,996

(22) Filed: Oct. 18, 2021

(51) Int. Cl. A61J 1/20

(52) U.S. Cl. CPC A61J 1/201; A61J 1/2048; A61J 1/10

(57) ABSTRACT

In some embodiments, systems, apparatuses, and methods are provided herein useful to medical devices. In some embodiments, a medical device comprises a main body portion, the main body portion comprising a medication dispenser, a connection portion, wherein the connection portion is secured to a top of the main body portion, the connection portion comprising a needle, wherein the needle is located within a housing, and wherein the needle is communicatively coupled to the medication dispenser, the housing, wherein the housing includes a needle guard, wherein an upper surface of the needle guard extends beyond the needle, and a cover, wherein the cover is configured to seat on the connection portion, the cover comprising an outer cover, and an inner cover, wherein the inner cover is configured to seat on the needle, and wherein the inner cover and the outer cover form a single structure.

### TECHNICAL FIELD

This disclosure relates generally to the medical field and, more specifically, medical devices.

### BACKGROUND

Many medications and medicinal compounds are administered to patients parenterally. Oftentimes, the medicinal compounds are packaged separately from an intended delivery system (e.g., a medical bag). Components of medicinal compounds (e.g., medications, infusion solution, etc.) are often packaged separately. Components of medicinal compounds are often packaged separately to aid in maintaining the stability of the components of the medicinal compounds, reduce the complexity of the medicinal compounds and delivery systems, and decrease the volume of inventories materials. The components can be packaged, for example, in prefilled syringes, vials, ampuls, and large volume infusion solutions.

In use, the contents of one or more packages (e.g., prefilled syringes, vials, etc.) are combined in a medical bag. The mixing of the components (e.g., the medications infusion solutions, etc.) can occur in a pharmacy (e.g., in a medical facility, such as a hospital), at the point-of-care, in an outpatient facility, and/or during emergency transport.

A variety of approaches, and associated devices, currently exist for combining the components and readying the delivery system. One such approach includes a mating or transfer device that connects a packaging (e.g., a vial) with a medical bag. Another approach is the use of an adapter affixed to a needleless syringe in which the adapter mates with the medical bag. A third approach utilizes proprietary packaging and medical bags (i.e., packaging, such as syringes or vials and medical bags that utilize proprietary connectors).

While each of these approaches can be used to successfully combining the components and readying the delivery system, each has drawbacks. For example, use of a mating or transfer device, or an adapter and a needleless syringe, requires additional inventory. That is, medical providers (e.g., hospitals, medical offices, emergency vehicles, etc.) must maintain an inventory of the mating or transfer devices, adaptors, and/or needleless syringes. The use of proprietary packaging and medical bags requires medical providers to purchase and include in inventory such proprietary packaging and medical bags.

Further, the current approaches typically require that the entire quantity of the packaging be combined in the delivery system. For example, the current devices do not allow for a user to easily administer only a portion of the quantity of the packaging in a controlled manner. Additionally, the current approaches do not have a mechanism for quickly confirming that the contents of the packaging have been added to the delivery system.

Accordingly, a need exists for medical devices that can accurately deliver the correct quantity of medication while minimizing the risks of misadministration of the medication in a convenient and cost-effective manner.

### BRIEF DESCRIPTION OF THE DRAWINGS

Disclosed herein are embodiments of systems, apparatuses, and methods pertaining medical devices. This description includes drawings, wherein:

FIG. 1 depicts a medical system 100 including a medical device 110 and a medical bag 104, according to some embodiments;

FIG. 2A depicts a medical device 200, according to some embodiments;

FIG. 2B is an exploded view of the medical device 200 depicting a cover 220, a needle 202, and a needle guard 208, according to some embodiments;

FIG. 2C is an exploded view of the medical device 200 depicting a stopper retention 212 and a stopper 216, according to some embodiments;

FIG. 2D depicts a portion of the medical device 200, according to some embodiments;

FIG. 2E depicts a plunger rod 218 of the medical device 200, according to some embodiments;

FIG. 3 depicts a medical system 300 including a medical device 316 and a medical bag 304, according to some embodiments;

FIG. 4A depicts a medical device 400, according to some embodiments;

FIG. 4B depicts the medical device 400 in a dispensed state, according to some embodiments; and

FIG. 4C is a top view of the medical device 400, according to some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

Generally speaking, pursuant to various embodiments, systems, apparatuses and methods are provided herein pertaining to medical devices. In some embodiments, a medical device comprises a main body portion, the main body portion comprising a medication dispenser, a connection portion, wherein the connection portion is secured to a top of the main body portion, the connection portion comprising a needle, wherein the needle is located within a housing, and wherein the needle is communicatively coupled to the medication dispenser, the housing, wherein the housing includes a needle guard, wherein an upper surface of the needle guard extends beyond the needle, and a cover, wherein the cover is configured to seat on the connection portion, the cover comprising an outer cover, and an inner cover, wherein the inner cover is configured to seat on the needle, and wherein the inner cover and the outer cover form a single structure.

As previously discussed, precision can be critically important in the medical field, for example, with respect to medication dosage and delivery. One area in which precision is critical is in the administration of medication. While some medications are administered orally, others are administered via medical devices, such as syringes. These medications can be delivered directly to the patient (e.g., intravenously) or indirectly to the patient (e.g., via a medical bag). Typically, medications that are delivered indirectly to patients are of a higher concentration than those delivered directly and thus are diluted (in the case of liquids) or reconstituted (in the case of powders) before they reach the patient. Because medications that are delivered indirectly to patients are typically of a higher concentration, such medications should not be administered directly to a patient. Though this is known, situations arise in which a medication of a high concentration may be inadvertently delivered to a patient directly. Currently systems exist that help avoid such inadvertent delivery of highly concentrated medication. However, these systems typically utilize proprietary connectors. Such proprietary connectors are undesirable, as the use of such proprietary connectors may force a healthcare facility, or system, to adopt only a single style of medical equipment that includes, or is adapted to work with, the proprietary connectors. This can be costly for a healthcare facility, or system, and result in funds being used to purchase proprietary equipment which could be otherwise used on patient care.

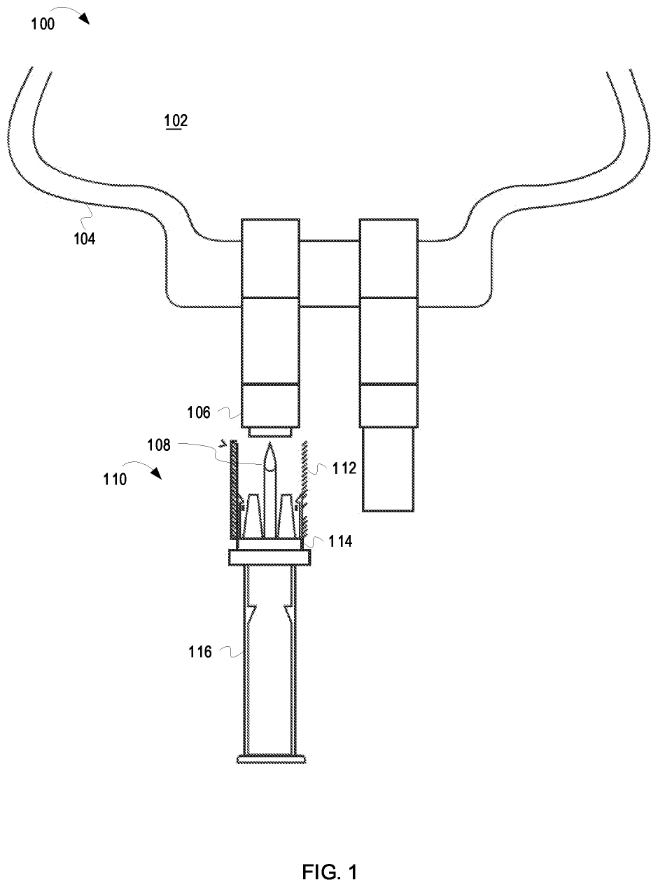

Described herein are systems, methods, and apparatuses that seek to minimize, if not eliminate, these drawbacks. In one embodiment, a medical device is provided that can be used for concentrated medications and does not require a proprietary connector. For example, such medical devices can be used with generic medical bags to administer concentrated medication to a patient via the generic medical bag. Additionally, in some embodiments, the medical device includes mechanisms by which inadvertent direct administration of concentrated medications can be avoided. For example, in one embodiment, the medical device includes a needle guard that would make direct administration of a medication contained in the medical device difficult and/or inconvenient. Additionally, the medical devices described herein can be used with generic medical bags, minimizing or eliminating the need for healthcare facilities, and systems, to adopt only a single style of medical equipment that includes, or is adapted to work with, proprietary medical devices. The discussion of FIG. 1 provides an overview of a medical system including such a medical device.

FIG. 1 depicts a medical system 100 including a medical device 110 and a medical bag 104, according to some embodiments. The medical bag can be of any suitable type, such as an intravenous (IV) bag. The medical bag 104 includes a fluid cavity 102 and a connector 106. The medical bag 104 can be of any type and, in some embodiments, is generally a generic medical bag (i.e., a medical bag without any special and/or proprietary features or fittings). The fluid cavity 102 is configured to contain a fluid, such as saline and/or medication (e.g., a concentrated medication, also referred to as a “concentrated solution medication” comprising a liquid and/or powder). The connector 106 is communicatively coupled to the fluid cavity 102, such that fluid, such as medication, can be added to the fluid cavity 102 via the connector 106. The connector 106 is configured to mate with the medical device 110.

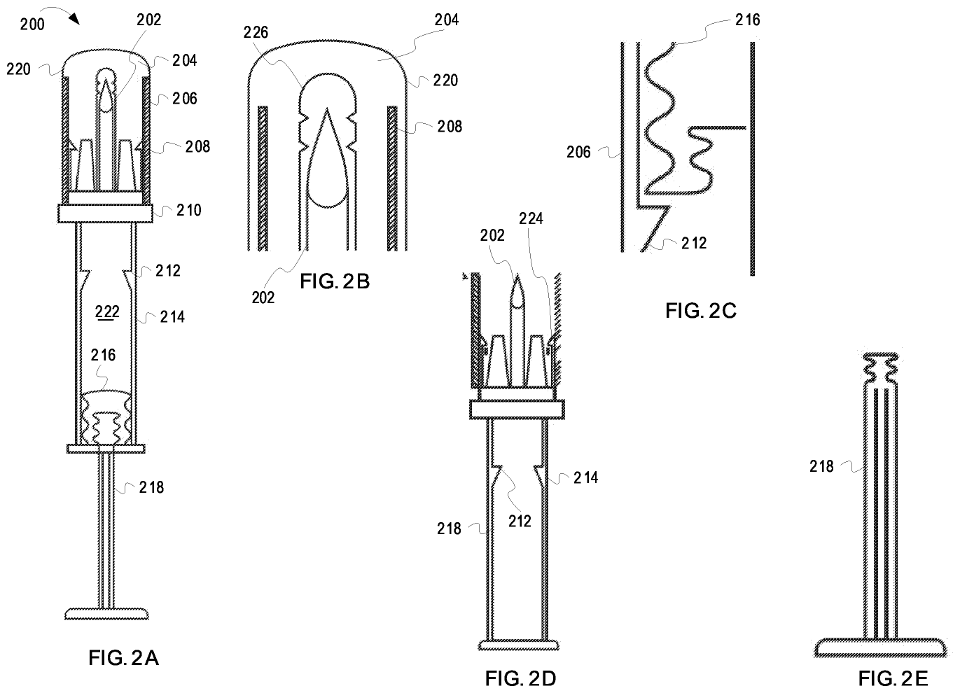

In the example depicted in FIG. 1, the medical device 110 is a syringe-type medical device. The medical device 110 generally comprises a main body portion 116, a connection portion 114, and a cover (not shown in FIG. 1). The main body portion 116 includes a medication dispenser. The medication dispenser is configured to hold, and dispense, fluid (e.g., medication). The connection portion 114 is secured to the top of the main body portion 116. For example, the connection portion 114 can be a separate unit that is connected to the main body portion 116 (e.g., via a threaded mating, clips, detents, etc.) or the connection portion 114 and the main body portion 116 can be manufactured as a single unit. The connection portion 114 generally includes a needle 108 and a housing (indicated by reference numeral 202 in FIGS. 2A and 2B). The needle 108 can take any suitable form and be comprised of any suitable material or combinations of materials. For example, the needle 108 may be a traditional needle, transfer spike, etc. and can be made of one or more of metal, plastic, etc. The needle 108 is communicatively coupled to the medication dispenser such that fluid contained in the medication dispenser can be expressed through the needle 108. The housing includes a needle guard 112. In one embodiment, an upper surface of the needle guard 112 extends beyond the needle 108. That is, in such embodiments, a tip of the needle 108 is lower than the upper surface of the needle guard 112 such that the needle 108 does not protrude beyond the needle guard 112. In such embodiments, the needle guard 112 can prevent accidental needle sticks and/or inadvertent direct parenteral administration of the fluid contained in the medication dispenser.

As previously discussed, in some embodiments, the medical bag 104 is a generic medical bag. Because the medical device 110 includes a needle 108 that is communicatively coupled to the medication dispenser, the medical device 110 can interface with a generic medical bag to administer fluid contained in the medication dispenser into the medical bag 104 via the needle 108. That is, due to the design of the connection portion, a proprietary or special medical bag is not required for use with the medical device 110. Such systems can be advantageous in that they do not require healthcare facilities, or systems, to adopt only a single style or brand of medical equipment, resulting in potential cost savings and convenience for the healthcare facilities, or systems.

While the discussion of FIG. 1 provides an overview of a medical system including a medical bag and a medical device, the discussion of FIGS. 2A-2E provides additional detail regarding the medical device.

FIGS. 2A-2E depict various views and/or features of a medical device 200 as described herein, according to some embodiments. As depicted in FIGS. 2A-2E, the medical device 200 is a syringe-type medical device. The medical device 200 generally comprises a main body portion 214, a connection portion 210, and a cover 220. The main body portion includes a medication dispenser 222. In one embodiment, the medication dispenser 222 is a hollow chamber within the main body portion 214. The medication dispenser 222 is configured to contain a fluid (e.g., a medication) and dispense the medication via a needle 202. In one embodiment, and as best depicted in FIGS. 2C and 2D, the main body portion 214 includes a stopper retention 212. The stopper retention 212 is configured to engage a stopper 216 of the plunger when the plunger is in a dispensed position (i.e., when the plunger has been depressed such that the fluid contained in the medication dispenser 222 has been expressed via the needle 202). In such embodiments, the stopper 216 and/or plunger rod 218 can be maintained in the dispensed position, making it easily verifiable that the contents of the medication dispenser 222 have been expressed and/or fully expressed. Additionally, the stopper retention 212 can prevent the plunger rod 218 from being moved from the dispensed position to the undispensed position and drawing fluid from the medical bag via the medical device 200.

The connection portion 210 generally comprises the needle 202 and a housing 206. The needle 202 is located within the housing 206 and communicatively coupled to the medication dispenser 222. The housing 206 includes a needle guard 208. In one embodiment, an upper surface of the needle guard 208 extends beyond the needle 202. In such embodiments, the needle guard 208 can aid in preventing needle sticks and/or inadvertent direct administration of the contents of the medication dispenser 222. The needle guard 208 can be made of any suitable material (e.g., rubber, plastic, etc.) and be dimensioned as desired to aid in preventing needle sticks and/or inadvertent direct administration of the contents of the medication dispenser 222. In some embodiments, the connection portion 210 includes a mating mechanism 224. In such embodiments, the mating mechanism 224 can mate with a corresponding feature of a medical bag to secure the medical device 200 to the medical bag. The mating mechanism 224 can take any suitable form. In one embodiment, the mating mechanism is configured such that the medical device 200 can mate with a generic medical bag and/or a large variety of medical bags, minimizing or eliminating the need for a healthcare facility, or system, to adopt only a single style of medical equipment. Alternatively, the mating mechanism 224 can take a specialized form, such that the medical device 200 is configured to mate with a specific type and/or brand of medical bag.

The cover 220 generally comprises an outer cover 204 and an inner cover 226. The outer cover 204 is configured to seat on the connection portion 210 and cover at least a portion of the connection portion 210. In some embodiments, the outer cover 204 is secured to the connection portion 210. For example, the outer cover 204 and/or connection portion 210 can include fasteners (e.g., clips, detents, rings, etc.) that secure the outer cover 204 to the connection portion 210. Additionally, or alternatively, the outer cover 204 and/or connection portion 210 can be dimensioned such that the outer cover 204 is secured to the connection portion 210 via a friction fit. The inner cover 226 is configured to cover at least a portion of the needle 202. In one embodiment, the outer cover 204 and the inner cover 226 form a single structure. In such embodiments, the inner cover 226 may be integral to the outer cover 204 such that removal of the outer cover 204 causes removal of the inner cover 226. The outer cover 204 and the inner cover 226 can be made of any suitable material (e.g., plastic, rubber, etc.) and need not be made of the same material. For example, in one embodiment, the outer cover 204 can be made of plastic and the inner cover 226 can be made of rubber that is molded, or otherwise integrated, into the outer cover 204.

In some embodiments, the medical device 200 includes a plunger. The plunger includes the plunger rod 218 and the stopper 216. The plunger is manipulable from a first position (e.g., an undispensed position) and a second position (e.g., a dispensed position). When in the second position, at least a portion of the fluid contained in the medication dispenser 222 has been expressed via the needle 202. In some embodiments, the stopper 216 is detachably affixed to the plunger rod 218. For example, the stopper 216 can be affixed to the plunger rod 218 via a press-fit, via threads on one or more of the stopper 216 and the plunger rod 218, via one or more fasteners, etc. In such embodiments, the plunger rod 218 can be easily removed from the stopper 216 once the fluid contained in the medication dispenser 222 has been expressed. As previously discussed, in some embodiments, the medication dispenser 222 includes a stopper retention 212 that engages the stopper 216. The stopper retention 212 can secure the stopper 216 in the dispensed position. In embodiments where the stopper 216 is detachably affixed to the plunger rod 218, once the stopper retention 212 has secured the stopper 216 in the dispensed position, the plunger rod 218 can be removed from the stopper 216. Such embodiments may allow people, such as healthcare workers, to quickly and easily verify that the contents of the medication dispenser 222 have been expressed. Additionally, removal of the plunger rod 218 may allow more space for healthcare workers to perform their tasks as well as prevent inadvertent manipulation of the stopper from the dispensed position.

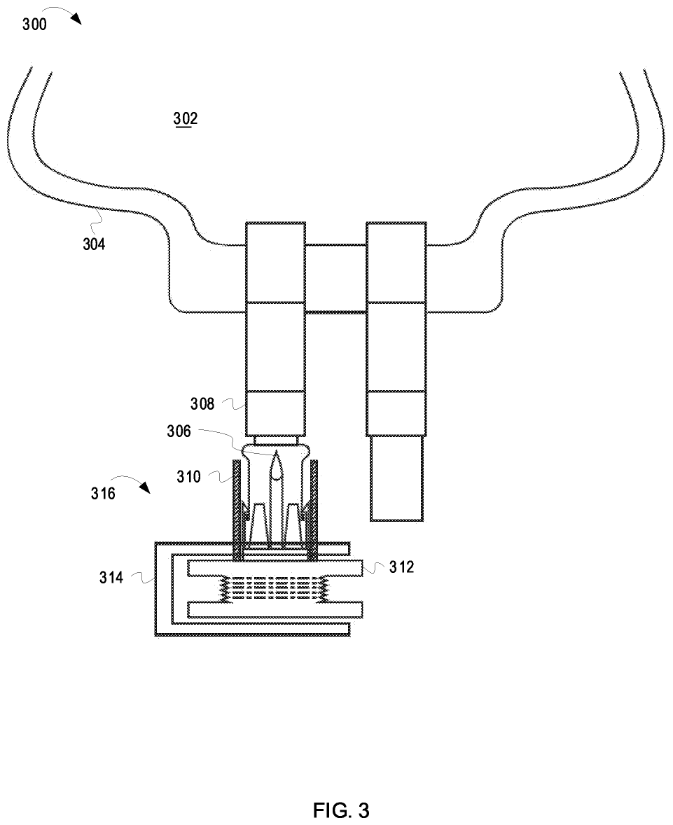

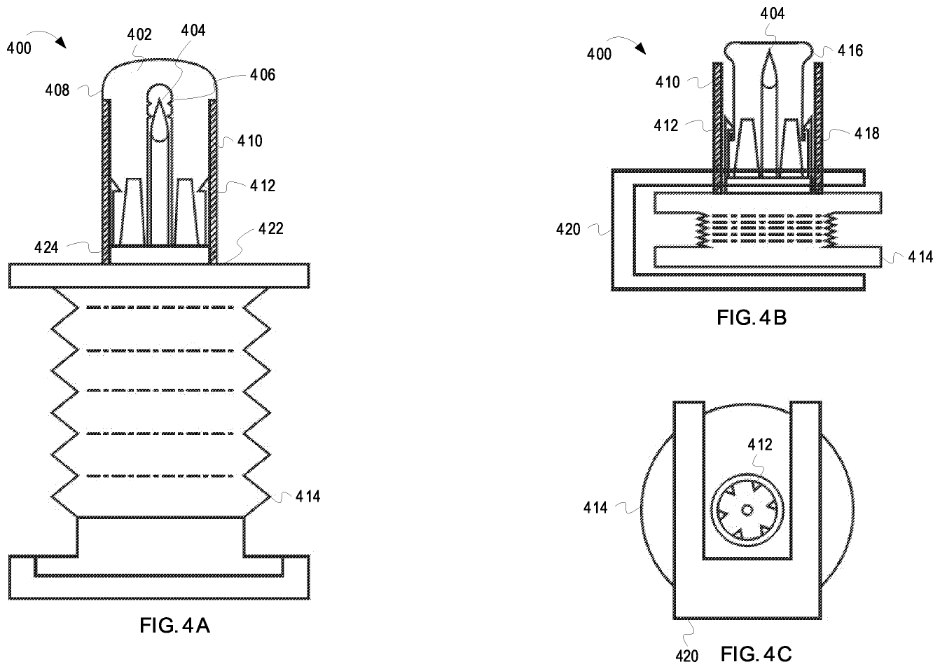

While the discussion of FIGS. 1 and 2A-2E provides additional detail regarding a medical system and a syringe-type medical device, the discussion of FIGS. 3 and 4A-4C provides additional detail regarding a medical system including a bellows-type medical device.

FIG. 3 depicts a medical system 300 including a medical device 316 and a medical bag 304, according to some embodiments. The medical bag 304 includes a fluid cavity 302 and a connector 308. The medical bag 304 can be of any type and, in some embodiments, is generally a generic medical bag (i.e., a medical bag without any special and/or proprietary features or fittings). Such systems can be advantageous in that they do not require healthcare facilities, or systems, to adopt only a single style or brand of medical equipment, resulting in potential cost savings and convenience for the healthcare facilities, or systems. The fluid cavity 302 is configured to contain a fluid, such as saline and/or medication. The connector 308 is communicatively coupled to the fluid cavity 302, such that fluid, such as medication, can be added to the fluid cavity 302 via the connector 308. The connector 308 is configured to mate with the medical device 316.

In the example depicted in FIG. 3, the medical device 316 is a bellows-type medical device. The medical device 316 generally comprises a main body portion 312, a connection portion 310, and a cover (not shown in FIG. 3). The main body portion 312 includes a medication dispenser. The medication dispenser is configured to hold, and dispense, fluid (e.g., medication). The connection portion 310 is secured to the top of the main body portion 312. For example, the connection portion 310 can be a separate unit that is connected to the main body portion 312 (e.g., via a threaded mating, clips, detents, etc.) or the connection portion 310 and the main body portion 312 can be manufactured as a single unit. The connection portion 310 generally includes a needle 306 and a housing. The needle 306 is communicatively coupled to the medication dispenser such that fluid contained in the medication dispenser can be expressed through the needle 306. The housing includes a needle guard (indicated by reference numeral 410 in FIGS. 4A and 4B). In one embodiment, an upper surface of the needle guard extends beyond the needle 306. That is, in such embodiments, a tip of the needle 306 is lower than the upper surface of the needle guard such that the needle 306 does not protrude beyond the needle guard. In such embodiments, the needle guard can prevent accidental needle sticks and/or inadvertent direct administration of the fluid contained in the medication dispenser. In some embodiments, as depicted in the example provided in FIG. 3, the medical device 316 includes a clamp 314. The clamp 314 is configured to maintain the medical device 316 (i.e., the medication dispenser) in a dispensed state. In such embodiments, the clamp 314 can serve as a visual indicator that any fluid contained in the medication dispenser has been expressed via the needle 306. Additionally, in some embodiments, the clamp 314 can prevent the medical device 316 (i.e., the medication dispenser) from moving from the dispensed state and drawing fluid from the fluid cavity 302.

While the discussion of FIG. 3 provides an overview of a medical system including a medical bag and a medical device, the discussion of FIGS. 4A-4C provides additional detail regarding the medical device.

FIGS. 4A-4C depict various views and/or features of a medical device 400 as described herein, according to some embodiments. As depicted in FIGS. 4A-4C, the medical device 400 is a bellows-type medical device. The medical device 400 generally comprises a main body portion 414, a connection portion 422, and a cover 402. The main body portion 414 includes a medication dispenser. The medication dispenser is configured to house, and dispense, a fluid for dilution or a powder for reconstitution (e.g., medication). For example, the medication dispenser can dispense the fluid via a needle 404.

The connection portion 422 generally comprises the needle 404 and a housing 424. The needle 404 is communicatively coupled to the medication dispenser. That is, the fluid contained in the medication dispenser can be expressed via the needle 404. The housing 424 includes a needle guard 410. In one embodiment, an upper surface of the needle guard 410 extends beyond the needle 404. In such embodiments, the needle guard 410 can aid in preventing needle sticks and/or inadvertent direct administration of the contents of the medication dispenser. The needle guard 410 can be made of any suitable material (e.g., rubber, plastic, etc.) and be dimensioned as desired to aid in preventing needle stick and/or inadvertent direct administration of the contents of the medication dispenser. In some embodiments, the connection portion 422 includes a mating mechanism 412. In such embodiments, the mating mechanism 412 can mate with a corresponding feature of a medical bag to secure the medical device 400 to the medical bag. The mating mechanism 412 can take any suitable form. In one embodiment, the mating mechanism 412 is configured such that the medical device 400 can mate with a generic medical bag and/or a large variety of medical bags. Such systems can be advantageous in that they do not require healthcare facilities, or systems, to adopt only a single style or brand of medical equipment, resulting in potential cost savings and convenience for the healthcare facilities, or systems. Alternatively, the mating mechanism 412 can take a specialized form, such that the medical device 400 is configured to mate with a specific type and/or brand of medical bag. As depicted in FIG. 4B, the needle 404 is engaging a connector 416 of a medical bag (not shown) such that the connector 416 is communicatively coupled to the needle 404. The connector 416 is communicatively coupled to the medical bag (i.e., a fluid cavity of the medical bag). Because the needle 404 is communicatively coupled to the medication dispenser, when the needle 404 engages the connector 416, the medication dispenser is communicatively coupled to the fluid cavity of the medical bag.

The cover 402 generally comprises an outer cover 408 and an inner cover 406. The outer cover 408 is configured to seat on the connection portion 422 and cover at least a portion of the connection portion 422. In some embodiments, the outer cover 408 is secured to the connection portion 422. For example, the outer cover 408 and/or connection portion 422 can include fasteners (e.g., clips, detents, rings, etc.) that secure the outer cover 408 to the connection portion 422. Additionally, or alternatively, the outer cover 408 and/or connection portion 422 can be dimensioned such that the outer cover 408 is secured to the connection portion 422 via a friction fit. The inner cover 406 is configured to cover at least a portion of the needle 404. In one embodiment, the outer cover 408 and the inner cover 406 form a single structure. In such embodiments, the inner cover 406 may be integral to the outer cover 408 such that removal of the outer cover 408 causes removal of the inner cover 406. The outer cover 408 and the inner cover 406 can be made of any suitable material (e.g., plastic, rubber, etc.) and need not be made of the same material. For example, in one embodiment, the outer cover 408 can be made of plastic and the inner cover 406 can be made of rubber that is molded, or otherwise integrated, into the outer cover 408.

The medical device 400 is manipulable from a first position (e.g., an undispensed position) to a second position (e.g., a dispensed position). For example, with a bellows-type medical device, the manipulation of the medical device 400 from the first position to the second position causes the medication dispenser to dispense the fluid contained in the medication dispenser. In the example provided, the medical device 400 is in the first position in FIG. 4A and in the second position in FIG. 4B. The medical device 400 allows the mixing of the powder or liquid contents of the medication with the diluent in a medical bag via manipulation of the medical device 400 and/or the medical bag (e.g., applying and/or removing pressure from the medical device 400 and/or medical bag). In some embodiments, and as depicted in FIGS. 4B and 4C, the medical device 400 includes a clamp 420. The clamp seats on the main body portion 414 and/or the connection portion 422 and maintains the medical device 400 in the second position. That is, the clamp 420 can be applied to the main body portion 414 and/or the connection portion 422 after the medical device has been manipulated to the dispensed state. In such embodiments, the clamp can, in addition to maintaining the medical device 400 in the second position, provide a quick and easy visual reference that the fluid contained within the medication dispenser has been expressed. Additionally, clamp 420 may prevent inadvertent manipulation of the medical device from the dispensed position.

In some embodiments, a medical device comprises a main body portion, the main body portion comprising a medication dispenser, a connection portion, wherein the connection portion is secured to a top of the main body portion, the connection portion comprising a needle, wherein the needle is located within a housing, and wherein the needle is communicatively coupled to the medication dispenser, the housing, wherein the housing includes a needle guard, wherein an upper surface of the needle guard extends beyond the needle, and a cover, wherein the cover is configured to seat on the connection portion, the cover comprising an outer cover, and an inner cover, wherein the inner cover is configured to seat on the needle, and wherein the inner cover and the outer cover form a single structure.

In some embodiments, a medical device comprises a main body portion, the main body portion comprising a medication dispenser and a stopper retention, a connection portion, wherein the connection portion is secured to a top of the main body portion, the connection portion comprising a needle, wherein the needle is located within a housing, and wherein the needle is communicatively coupled to the medication dispenser, the housing wherein the housing includes a needle guard, wherein an upper surface of the needle guard extends beyond the needle, and a plunger, wherein the plunger is manipulable from a first position to a second position, wherein when manipulated from the first position to the second position medication housed in the medication dispenser is expressed via the needle, the plunger comprising a plunger rod, and a stopper, wherein the stopper is detachably affixed to the plunger rod, and wherein the stopper engages with the stopper retention when the plunger is in the second position to secure the stopper in the second position.

In some embodiments a medical system comprises a medical device, wherein the medical device comprises a main body portion, the main body portion comprising a medication dispenser, a connection portion, wherein the connection portion is secured to a top of the main body portion, the connection portion comprising a needle, wherein the needle is communicatively coupled to the medication dispenser, the housing, wherein the housing includes a needle guard, wherein an upper surface of the needle guard extends beyond the needle, and a cover, wherein the cover is configured to seat on the connection portion, the cover comprising an outer cover, and an inner cover, wherein the inner cover is configured to seat on the needle, and wherein the inner cover and the outer cover form a single structure, and a medical bag, wherein the medical bag comprises a fluid cavity, and a connector, wherein the connector is communicatively coupled to the fluid cavity, and wherein the connector is configured to mate with the connection portion of the medical device.

Those skilled in the art will recognize that a wide variety of other modifications, alterations, and combinations can also be made with respect to the above described embodiments without departing from the scope of the disclosure, and that such modifications, alterations, and combinations are to be viewed as being within the ambit of the inventive concept.

What is claimed is:

1. A medical device, the medical device comprising:
a main body portion, the main body portion comprising:
a medication dispenser;
a connection portion, wherein the connection portion is secured to a top of the main body portion, the connection portion comprising:
a needle, wherein the needle is located within a housing, and wherein the needle is communicatively coupled to the medication dispenser;
the housing, wherein the housing includes a needle guard, wherein an upper surface of the needle guard extends beyond the needle; and
a cover, wherein the cover is configured to seat on the connection portion, the cover comprising:
an outer cover; and
an inner cover, wherein the inner cover is configured to seat on the needle, and wherein the inner cover and the outer cover form a single structure.

2. The medical device of claim 1, wherein the medication dispenser is one of a syringe and a bellows.

3. The medical device of claim 2, wherein the medication dispenser is the syringe, wherein the syringe includes a plunger including a stopper and a plunger rod, wherein the plunger rod is removable from the stopper.

4. The medical device of claim 3, wherein the medication dispenser includes a stopper retention, wherein the stopper retention is configured to maintain the stopper at a dispensed location.

5. The medical device of claim 2, wherein the medication dispenser is the bellows, and further comprising:
a clamp, wherein the clamp is configured to maintain the bellows in a dispensed state.

6. The medical device of claim 1, wherein the connection portion is configured to interface with a medical bag.

7. The medical device of claim 6, wherein the connection portion includes a mating mechanism, wherein the mating mechanism is configured to mate with a complementary mating mechanism of the medical bag.

8. The medical device of claim 1, wherein the medication dispenser contains a concentrated medication.

9. A medical device, the medical device comprising:
a main body portion, the main body portion comprising:
a medication dispenser; and
a stopper retention;
a connection portion, wherein the connection portion is secured to a top of the main body portion, the connection portion comprising:
a needle, wherein the needle is located within a housing, and wherein the needle is communicatively coupled to the medication dispenser;
the housing, wherein the housing includes a needle guard, wherein an upper surface of the needle guard extends beyond the needle; and
a plunger, wherein the plunger is manipulable from a first position to a second position, wherein when manipulated from the first position to the second position medication housed in the medication dispenser is expressed via the needle, the plunger comprising:
a plunger rod; and
a stopper, wherein the stopper is detachably affixed to the plunger rod, and wherein the stopper engages with the stopper retention when the plunger is in the second position to secure the stopper in the second position.

10. The medical device of claim 9, wherein the stopper retention includes a ledge proximal to the connection portion, and wherein the stopper retention includes an angled sidewall extending away from the connection portion.

11. The medical device of claim 9, wherein the connection portion includes a mating mechanism, and wherein the mating mechanism is configured to mate with a complementary mating mechanism of a medical bag.

12. The medical device of claim 9, wherein the medication dispenser contains a concentrated medication.

13. The medical device of claim 9, wherein the plunger rod comprises plastic and the stopper comprises rubber, and wherein the plunger rod is press-fitted into the stopper.

14. The medical device of claim 9, wherein the medication dispenser contains a concentrated medication.

15. A medical system, the medical system comprising:
a medical device, wherein the medical device comprises:
a main body portion, the main body portion comprising:
a medication dispenser;
a connection portion, wherein the connection portion is secured to a top of the main body portion, the connection portion comprising:
a needle, wherein the needle is located within a housing, and wherein the needle is communicatively coupled to the medication dispenser;
the housing, wherein the housing includes a needle guard, wherein an upper surface of the needle guard extends beyond the needle; and
a cover, wherein the cover is configured to seat on the connection portion, the cover comprising:
an outer cover; and
an inner cover, wherein the inner cover is configured to seat on the needle, and wherein the inner cover and the outer cover form a single structure; and
a medical bag, wherein the medical bag comprises:
a fluid cavity; and
a connector, wherein the connector is communicatively coupled to the fluid cavity, and wherein the connector is configured to mate with the connection portion of the medical device.

16. The medical system of claim 15, wherein the medication dispenser is one of a syringe and a bellows.

17. The medical system of claim 16, wherein the medication dispenser is the syringe, wherein the syringe includes a plunger including a stopper and a plunger rod, wherein the plunger rod is removable from the stopper.

18. The medication system of claim 17, wherein the medication dispenser includes a stopper retention, wherein the stopper retention is configured to maintain the stopper at a dispensed location.

19. The medical system of claim 16, wherein the medication dispenser is the bellows, and further comprising:
a clamp, wherein the clamp is configured to maintain the bellows in a dispensed state.

20. The medical system of claim 15, wherein the medication dispenser contains a concentrated medication and wherein the fluid cavity contains a medical fluid.
